(19) United States
(12) Patent Application Publication
(10) Pub. No.: US 2026/0008737 A1
(43) Pub. Date: Jan. 8, 2026

(54) CATALYSTS FOR ISOBUTANOL SYNTHESIS

(71) Applicants: UOP LLC, Des Plaines, IL (US); CHINA PETROLEUM & CHEMICAL CORPORATION, Beijing (CN)

(72) Inventors: Richard Long, Katy, TX (US); Desmond Schipper, Katy, TX (US); Tian Ruan, Katy, TX (US)

(21) Appl. No.: 18/881,781

(22) PCT Filed: Jul. 12, 2022
(86) PCT No.: PCT/US2022/073631

§ 371 Date: Jan. 7, 2025

(51) Int. Cl. C07C 29/34; B01J 21/08; B01J 21/10; B01J 23/72; B01J 37/02; B01J 37/03

(52) U.S. Cl. CPC C07C 29/34; B01J 21/08; B01J 21/10; B01J 23/72; B01J 37/0201; B01J 37/031

(57) ABSTRACT

M/M²⁺M⁴⁺ oxide catalysts have been developed for use in producing isobutanol in propanol-methanol, ethanol-methanol and propanol/ethanol mixture-methanol reactions. The catalysts can also be used in making n-butanol in ethanol-ethanol reactions. M may comprise one or more metals from Groups 3-12 of the Periodic Table. M²⁺ may comprise divalent magnesium, calcium, strontium, barium, or combinations thereof. M⁴⁺ may comprise tetravalent silicon, titanium, zirconium, or combinations thereof. Catalysts, and methods of using the catalysts are described.

### BACKGROUND

Isobutanol is an organic solvent and a feedstock in the manufacturing of isobutyl acetate and isobutyl esters. It can also be blended directly with gasoline to improve octane number and combustion efficiency or be used neat as an alternative fuel. Isobutanol has relatively higher energy density and lower volatility compared to ethanol. In addition, it does not readily absorb water from air, preventing or reducing the corrosion of engines and pipelines. Although isobutanol has many potential uses, its synthesis is limited. Isobutanol is currently produced through the carbonylation of propylene. This process involves reacting propylene with carbon monoxide and hydrogen to generate butyraldehyde and isobutyraldehyde, hydrogenating them to n-butanol and isobutanol, followed by separation of the butanols. A new alternative technology is biomass fermentation. However, isobutanol selectivities in these two homogeneous processes are low, and the productivities are limited, resulting in a high cost of isobutanol.

Guerbet reaction is an alternative process for isobutanol synthesis from methanol and ethanol/propanol. This reaction is of special importance because it can produce value added isobutanol from the low-cost mixed alcohols. The Guerbet reaction takes place by a coupling process between alcohols on multi-functional catalysts with dehydrogenation activity, strong surface basicity, mild acidity, and hydrogenation activity. The reactions are below:

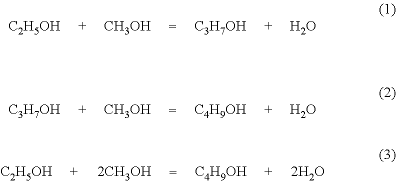

As a result, various catalysts and processes for producing isobutanol from methanol, ethanol and propanol have been sought. For example, U.S. Pat. Nos. 5,581,602, 5,707,920, 5,770,541, 5,908,807, 5,939,352 and 6,034,141 describe precious metal loaded alkali metal doped ZnMnZr oxide catalysts for converting methanol and ethanol, or methanol, ethanol and propanol to isobutanol.

U.S. Pat. No. 5,559,275 discloses a process for the conversion of methanol, ethanol and propanol to higher branched oxygenates, such as isobutanol on a catalyst comprising a) a mixed oxide support having at least two components selected from Zn, Mg, Zr, Mn, Ti, Cr and La oxides: and b) an active metal selected from Pd, Pt, Ag, Rh, Co, and mixtures thereof.

Carlini, “Guerbet condensation of methanol with n-propanol to isobutyl alcohol over heterogeneous bifunctional catalysts based on Mg-Al mixed oxides partially substituted by different metal components,” Journal of Molecular Catalysis A: Chemical, 2005, 232, 13, describes Pd, Rh, Ni and Cu doped on Mg-Al mixed oxides for isobutanol synthesis from methanol and propanol.

US 20190031585 discloses a method of converting ethanol to higher alcohols (such as n-butanol) on Cu-MgO-Al₂O₃ catalyst having less than 0.25 wt % Cu. The Cu is a pseudo-single-atom and it is small and highly dispersed on the support.

Gabriëls, “Review of catalytic systems and thermodynamics for the Guerbet condensation reaction and challenges for biomass valorization,” Catalysis Science & Technology, 2015, 5, 3876, summarizes a series of catalysts for the reaction between methanol and ethanol/propanol, including alkali or alkaline earth supported on Al₂O₃, Ca or Sr hydroxy apatite, hydrotalcite, MgO, Mg(OH)₂, Rb-Li exchanged zeolite X and Na₂CO₃/NaX.

There remains a need for catalysts producing isobutanol from methanol, ethanol, and propanol, and for methods of making and using the catalysts.

### DETAILED DESCRIPTION

M/M²⁺M⁴⁺ oxide catalysts have been developed which exhibit good isobutanol yield in propanol-methanol, ethanol-methanol and propanol/ethanol mixture-methanol reactions. They also exhibit good n-butanol yield in ethanol-ethanol reactions. Methanol and ethanol can be reacted to form propanol, which can then react with methanol to form isobutanol using the M/M²⁺M⁴⁺ oxide catalysts. Alternatively, methanol and propanol can be reacted directly to produce isobutanol using the M/M²⁺M⁴⁺ oxide catalysts. Mixtures of propanol and ethanol can be reacted with methanol. Ethanol can also be reacted with ethanol to form n-butanol.

One aspect of the invention is an alcohol condensation catalyst for the synthesis of isobutanol. In one embodiment, the catalyst comprises a M/M²⁺M⁴⁺ oxide catalyst. M may comprise one or more metals from Groups 3-12 of the Periodic Table. M²⁺ may comprise divalent magnesium, calcium, strontium, barium, or combinations thereof. M²⁺ may consist essentially of divalent magnesium, calcium, strontium, barium, or combinations thereof. M²⁺ may be selected from the group consisting of divalent magnesium, calcium, strontium, barium, or combinations thereof. M⁴⁺ may comprise tetravalent silicon, titanium, zirconium, or combinations thereof. M⁴⁺ may consist essentially of tetravalent silicon, titanium, zirconium, or combinations thereof. M⁴⁺ may be selected from the group consisting of tetravalent silicon, titanium, zirconium, or combinations thereof.

In some embodiments, M comprises Cu, Co, Fe, Ni, Ru, Rh, Pd, Ir, Pt, Ag, Au, or combinations thereof. In some embodiments, M is Cu.

In some embodiments M is present in the catalyst in an amount of 0.01 wt % to 50 wt %, or 0.01 wt % to 40 wt %, or 0.01 wt % to 30 wt %, or, 0.01 wt % to 25 wt %, or 0.01 wt % to 20 wt %, or 0.01 wt % to 15 wt %, or 0.01 wt % to 10 wt %, or 0.1 wt % to 50 wt %, or 0.1 wt % to 40 wt %, or 0.1 wt % to 30 wt %, or, 0.1 wt % to 25 wt %, or 0.1 wt % to 20 wt %, or 0. 1 wt % to 15 wt %, or 0.1 wt % to 10 wt %, or 0.5 wt % to 50 wt %, or 0.5 wt % to 40 wt %, or 0.5 wt % to 30 wt %, or, 0.5 wt % to 25 wt %, or 0.5 wt % to 20 wt %, or 0.5 wt % to 15 wt %, or 0.5 wt % to 10 wt %, or 1 wt % to 50 wt %, or 1 wt % to 40 wt %, or 1 wt % to 30 wt %, or, 1 wt % to 25 wt %, or 1 wt % to 20 wt %, or 1 wt % to 15 wt %, or 1 wt % to 10 wt %, or 2 wt % to 50 wt %, or 2 wt % to 40 wt %, or 2 wt % to 30 wt %, or, 2 wt % to 25 wt %, or 2 wt % to 20 wt %, or 2 wt % to 15 wt %, or 2 wt % to 10 wt %, or 5 wt % to 50 wt %, or 5 wt % to 40 wt %, or 5 wt % to 30 wt %, or, 5 wt % to 25 wt %, or 5 wt % to 20 wt %, or 5 wt % to 15 wt %, or 5 wt % to 10 wt %, or 10 wt % to 50 wt %, or 10 wt % to 40 wt %, or 10 wt % to 30 wt %, or, 10 wt % to 25 wt %, or 10 wt % to 20 wt %, or 10 wt % to 15 wt %, or 15 wt % to 50 wt %, or 15 wt % to 40 wt %, or 15 wt % to 30 wt %, or, 15 wt % to 25 wt %, or 15 wt % to 20 wt %, or 20 wt % to 50 wt %, or 20 wt % to 40 wt %, or 20 wt % to 30 wt %, or 20 wt % to 25 wt %, or 25 wt % to 50 wt %, or 25 wt % to 40 wt %, or 25 wt % to 30 wt %, or 30 wt % to 50 wt %, or 30 wt % to 40 wt %, or 35 wt % to 50 wt %, or 35 wt % to 40 wt %, or 40 wt % to 50 wt %.

In some embodiments, M2+O is present in the catalyst in an amount of 1 wt % to 50 wt %, or 1 wt % to 75 wt %, or 1 wt % to 70 wt %, or 1 wt % to 65 wt %, or 1 wt % to 60 wt %, or 2 wt % to 98 wt %, or 2 wt % to 75 wt %, or 2 wt % to 70 wt %, or 2 wt % to 65 wt %, or 2 wt % to 60 wt %, or 5 wt % to 98 wt %, or 5 wt % to 75 wt %, or 5 wt % to 70 wt %, or 5 wt % to 65 wt %, or 5 wt % to 60 wt %, or 10 wt % to 98 wt %, or 10 wt % to 75 wt %, or 10 wt % to 70 wt %, or 10 wt % to 65 wt %, or 10 wt % to 60 wt %, or 15 wt % to 98 wt %, or 15 wt % to 75 wt %, or 15 wt % to 70 wt %, or 15 wt % to 65 wt %, or 15 wt % to 60 wt %, or 20 wt % to 98 wt %, or 20 wt % to 75 wt %, or 20 wt % to 70 wt %, or 20 wt % to 65 wt %, or 20 wt % to 60 wt %, or 25 wt % to 98 wt %, or 25 wt % to 75 wt %, or 25 wt % to 70 wt %, or 25 wt % to 65 wt %, or 25 wt % to 60 wt %, or 30 wt % to 98 wt %, or 30 wt % to 75 wt %, or 30 wt % to 70 wt %, or 30 wt % to 65 wt %, or 30 wt % to 60 wt %, or 35 wt % to 98 wt %, or 35 wt % to 75 wt %, or 35 wt % to 70 wt %, or 35 wt % to 65 wt %, or 35 wt % to 60 wt %, or, 40 wt % to 98 wt %, or 40 wt % to 75 wt %, or 40 wt % to 70 wt %, or 40 wt % to 65 wt %, or 40 wt % to 60 wt %, or 45 wt % to 98 wt %, or 45 wt % to 75 wt %, or 45 wt % to 70 wt %, or 45 wt % to 65 wt %, or 45 wt % to 60 wt %, or 50 wt % to 98 wt %, or 50 wt % to 75 wt %, or 50 wt % to 70 wt %, or 50 wt % to 65 wt %, or 50 wt % to 60 wt %, or 55 wt % to 98 wt %, or 55 wt % to 75 wt %, or 55 wt % to 70 wt %, or 55 wt % to 65 wt %, or 55 wt % to 60 wt % 5 wt % to 98 wt %, or 5 wt % to 75 wt %, or 5 wt % to 70 wt %, or 5 wt % to 65 wt %, or 5 wt % to 60 wt %, or 10 wt % to 98 wt %, or 10 wt % to 75 wt %, or 10 wt % to 70 wt %, or 10 wt % to 65 wt %, or 10 wt % to 60 wt %, or 15 wt % to 98 wt %, or 15 wt % to 75 wt %, or 15 wt % to 70 wt %, or 15 wt % to 65 wt %, or 15 wt % to 60 wt %, or 20 wt % to 98 wt %, or 20 wt % to 75 wt %, or 20 wt % to 70 wt %, or 20 wt % to 65 wt %, or 20 wt % to 60 wt %, or 25 wt % to 98 wt %, or 25 wt % to 75 wt %, or 25 wt % to 70 wt %, or 25 wt % to 65 wt %, or 25 wt % to 60 wt %, or 30 wt % to 98 wt %, or 30 wt % to 75 wt %, or 30 wt % to 70 wt %, or 30 wt % to 65 wt %, or 30 wt % to 60 wt %, or 35 wt % to 98 wt %, or 35 wt % to 75 wt %, or 35 wt % to 70 wt %, or 35 wt % to 65 wt %, or 35 wt % to 60 wt %, or, 40 wt % to 98 wt %, or 40 wt % to 75 wt %, or 40 wt % to 70 wt %, or 40 wt % to 65 wt %, or 40 wt % to 60 wt %, or 45 wt % to 98 wt %, or 45 wt % to 75 wt %, or 45 wt % to 70 wt %, or 45 wt % to 65 wt %, or 45 wt % to 60 wt %, or 50 wt % to 98 wt %, or 50 wt % to 75 wt %, or 50 wt % to 70 wt %, or 50 wt % to 65 wt %, or 50 wt % to 60 wt %, or 55 wt % to 98 wt %, or 55 wt % to 75 wt %, or 55 wt % to 70 wt %, or 55 wt % to 65 wt %, or 55 wt % to 60 wt %.

In some embodiments, M⁴⁺O₂ is present in the catalyst in an amount of 1 wt % to 98 wt %, or 1 wt % to 75 wt %, or 1 wt % to 70 wt %, or 1 wt % to 65 wt %, or 1 wt % to 60 wt %, or 2 wt % to 98 wt %, or 2 wt % to 75 wt %, or 2 wt % to 70 wt %, or 2 wt % to 65 wt %, or 2 wt % to 60 wt %, or 5 wt % to 98 wt %, or 5 wt % to 75 wt %, or 5 wt % to 70 wt %, or 5 wt % to 65 wt %, or 5 wt % to 60 wt %, or 10 wt % to 98 wt %, or 10 wt % to 75 wt %, or 10 wt % to 70 wt %, or 10 wt % to 65 wt %, or 10 wt % to 60 wt %, or 15 wt % to 98 wt %, or 15 wt % to 75 wt %, or 15 wt % to 70 wt %, or 15 wt % to 65 wt %, or 15 wt % to 60 wt %, or 20 wt % to 98 wt %, or 20 wt % to 75 wt %, or 20 wt % to 70 wt %, or 20 wt % to 65 wt %, or 20 wt % to 60 wt %, or 25 wt % to 98 wt %, or 25 wt % to 75 wt %, or 25 wt % to 70 wt %, or 25 wt % to 65 wt %, or 25 wt % to 60 wt %, or 30 wt % to 98 wt %, or 30 wt % to 75 wt %, or 30 wt % to 70 wt %, or 30 wt % to 65 wt %, or 30 wt % to 60 wt %, or 35 wt % to 98 wt %, or 35 wt % to 75 wt %, or 35 wt % to 70 wt %, or 35 wt % to 65 wt %, or 35 wt % to 60 wt %, or, 40 wt % to 98 wt %, or 40 wt % to 75 wt %, or 40 wt % to 70 wt %, or 40 wt % to 65 wt %, or 40 wt % to 60 wt %, or 45 wt % to 98 wt %, or 45 wt % to 75 wt %, or 45 wt % to 70 wt %, or 45 wt % to 65 wt %, or 45 wt % to 60 wt %, or 50 wt % to 98 wt %, or 50 wt % to 75 wt %, or 50 wt % to 70 wt %, or 50 wt % to 65 wt %, or 50 wt % to 60 wt %, or 55 wt % to 98 wt %, or 55 wt % to 75 wt %, or 55 wt % to 70 wt %, or 55 wt % to 65 wt %, or 55 wt % to 60 wt %.

In some embodiments, M²⁺ comprises divalent magnesium.

In some embodiments, M⁴⁺ comprises silicon.

In some embodiments, the catalyst further comprises a salt or oxide of a metal from Group 1 of the Periodic Table, or combinations thereof. In some embodiments, the Group 1 metal comprises Li, Na, K, Rb, Cs, or combinations thereof. The Group 1 metal may be present in an amount of 0.01 wt % to 10 wt %, or 0.05 wt % to 10 wt %, or 0.1 wt % to 10 wt %, or 0.5 wt % to 10 wt %, or 1 wt % to 10 wt %, or 2 wt % to 10 wt %, or 3 wt % to 10 wt %, or 4 wt % to 10 wt %, or 5 wt % to 10 wt %, or 6 wt % to 10 wt %, or 7 wt % to 10 wt %, or 8 wt % to 10 wt %, or 9 wt % to 10 wt %, or 0.01 wt % to 8 wt %, or 0.01 wt % to 5 wt %, or 0.01 wt % to 2 wt %, or 0.01 wt % to 1 wt %, or 0.01 wt % to 0.5 wt %, or 0.05 wt % to 8 wt %, or 0.05 wt % to 5 wt %, or 0.05 wt % to 2 wt %, or 0.05 wt % to 1 wt %, or 0.05 wt % to 0.5 wt %, or 0.1 wt % to 8 wt %, or 0.1 wt % to 5 wt %, or 0.1 wt % to 2 wt %, or 0.1 wt % to 1 wt %, or 0.1 wt % to 0.5 wt %, or or 0.5 wt % to 8 wt %, or 0.5 wt % to 5 wt %, or 0.5 wt % to 2 wt %, or 0.5 wt % to 1 wt %, or 1 wt % to 8 wt %, or 1 wt % to 5 wt %, or 1 wt % to 2 wt %, or 2 wt % to 8 wt %, or 2 wt % to 5 wt %, or 3 wt % to 5 wt %, or 4 wt % to 8 wt %, or 4 wt % to 5 wt %.

Another aspect of the invention is a method of producing isobutanol or n-butanol. In one embodiment, the method comprises: reacting ethanol or propanol with methanol in the presence of an alcohol condensation catalyst under reaction conditions to produce isobutanol or reacting ethanol with ethanol in the presence of an alcohol condensation catalyst under reaction conditions to produce n-butanol; wherein the alcohol condensation catalyst comprises a M/M²⁺M⁴⁺ oxide catalyst: wherein M comprises metals from Groups 3-12 of the Periodic Table, or combinations thereof: wherein M²⁺ comprises divalent magnesium, calcium, strontium, barium, or combinations thereof: wherein M⁴⁺ comprises tetravalent silicon, titanium, zirconium, or combinations thereof.

The M/M²⁺M⁴⁺ oxide catalyst is described above.

In some embodiments, the reaction conditions comprise one or more of: a temperature in a range of 100° C. to 500° C.: or a pressure in a range of 5 kPa to 30,000 kPa.

The method of producing isobutanol or n-butanol with the M/M²⁺M⁴⁺ oxide catalyst allows good methanol, ethanol or propanol conversion, as well as good isobutanol or n-butanol selectivity and productivity.

### EXAMPLES

### Example 1

Mg₂SiO₄ was prepared by reacting Mg(NO₃)₂·6H₂O (dissolved in ethanol) with a Na₂SiO₃·9H₂O aqueous solution at 170° C. in autoclave for 24 hours, followed by filtering, washing, drying, and calcination at 450° C. for 4 hours. Cu was impregnated on the Mg₂SiO₄ surface by incipient wetness impregnation. This produced 5% Cu/Mg₂SiO₄ catalyst with a BET surface area of 203 m²/g and a pore volume of 0.21 cm³/g.

The catalyst performance testing was conducted in a micro-reactor under the conditions of 289-337° C., 600 psi, 11.4% propanol, 22.8% methanol, balance N₂ and a GHSV of 3000 ml/g-h. The testing results are summarized in Table 1. The methanol conversions were 28-42%, the propanol conversions were 43-66%, the isobutanol selectivities were 30-46%, and the isobutanol productivities were 154-361 g/kg-h under the testing conditions.

The above results indicate that Cu doped Mg₂SiO₄ catalyst is a good catalyst for converting propanol and methanol to isobutanol through Guerbet reaction.

### Example 2

Cu_{0.19}Ca_{1.81}ZrO₄ catalyst was prepared with a conventional co-precipitation method.

3.65 g Cu(NO₃)₂·2.5H₂O, 36.05 g Ca(NO₃)₂·4H₂O and 31.41 g ZrO(NO₃)₂·xH₂O were dissolved in 167 g deionized water in a beaker.

In a separate beaker, 52.39 g K₂CO₃ were dissolved in 188 g deionized water.

The two solutions were pumped to a third beaker containing 200 g deionized water at 70° C. with stirring. The pH value of the mixture was maintained at 7.0. After the co-precipitation process was complete, the mixture was stirred for an additional one hour.

Subsequently, the slurry was filtered and washed with deionized water three times. The obtained paste was dried at 120° C. for 12 hours and calcined at 600° C. for 4 hours.

1.00 g sample was loaded into a catalyst basket and placed in a stainless steel autoclave along with 29 g solution with 2:1 CH₃OH-C₃H₇OH molar ratio. The autoclave was sealed, charged with 2200 psi of ultra-high purity N₂, and vented after two hours to give a sealed system with an N₂ headspace. The autoclave was heated to 325° C. at a 2° C./min ramp rate with stirring and held at 325° C. for 15 hours before cooling to room temperature. The liquid, catalyst basket, and autoclave weights were recorded. The liquid was analyzed by GC to provide information about methanol and propanol conversions and isobutanol productivity. 55% methanol conversion, 46% propanol conversion, 28% isobutanol selectivity and 148 g/kg-h isobutanol productivity were achieved.

### Example 3

5% Cu/Mg3Si₄O₁₁ was prepared with precipitation followed by impregnation. 25.6 g Mg(NO₃)₂·6H₂O and 3.81 g acetic acid were dissolved in 50 ml deionized water in a 500 ml Nalgene bottle. 37.9 g Na₂SiO₃·9H₂O was dissolved in 300 ml deionized water and then added rapidly to the Mg(NO₃)₂ solution with stirring. The mixture was stirred vigorously for 30 min and then allowed to stand sealed for four days at room temperature. Next the slurry was filtered, washed with deionized water, and dried at 120° C. The dried solid was mixed with 100 ml H₂O in a 200 ml Teflon lined autoclave and heated for 24 h at 200° C. The solid was filtered and washed with 500 ml H₂O, dried, and calcined at 500° C. for 4 h.

8 g of the calcined material (Mg₃Si₄O₁₁) was impregnated with a solution of 0.541 g Cu(NO₃)₂·2.5 H₂O in 2.8 ml H₂O, and the material was calcined at 400° C. for 8 h.

1.00 g sample was loaded into a catalyst basket and placed in a stainless steel autoclave along with 29 g solution with 2:1 CH₃OH-C₃H₇OH molar ratio. The autoclave was sealed, charged with 2200 psi of ultra-high purity N₂, and vented after two hours to give a sealed system with an N₂ headspace. The autoclave was heated to 325° C. at a 2° C./min ramp rate with stirring and held at 325° C. for 15 hours before cooling to room temperature. The liquid, catalyst basket, and autoclave weights were recorded. The liquid was analyzed by GC to provide information about methanol and propanol conversions and isobutanol productivity. 55% methanol conversion, 60% propanol conversion, 30% isobutanol selectivity, and 206 g/kg-h isobutanol productivity were achieved.

### Example 4

1.0 g 5% Cu/Mg₃Si₄O₁₁ sample from EXAMPLE 3 was loaded into a catalyst basket and placed in a stainless steel autoclave along with 31.3 g ethanol solution. The autoclave was sealed, charged with 2200 psi of ultra-high purity N₂, and vented after two hours to give a sealed system with an N₂ headspace. The autoclave was heated to 325° C. at a 2° C./min ramp rate with stirring and held at 325° C. for 15 hours before cooling to room temperature. The liquid, catalyst basket, and autoclave weights were recorded. The liquid was analyzed by GC to provide information about ethanol conversion and n-butanol productivity. 72% ethanol conversion, 22% n-butanol selectivity and 263 g/kg-h n-butanol productivity were achieved.

TABLE 1
Isobutanol synthesis from propanol-methanol
reaction on the catalyst in Example 1

| | | | | Isobutanol |
|---|---|---|---|---|
| | Methanol | Propanol | Isobutanol | productivity |
| T (° C.) | conversion | conversion | selectivity | (g/kg-h) |
| 289 | 28% | 43% | 30% | 154 |
| 313 | 36% | 53% | 43% | 269 |
| 337 | 42% | 66% | 46% | 361 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. An alcohol condensation catalyst for the synthesis of isobutanol comprising:
a M/M²⁺M⁴⁺ oxide catalyst;
wherein M comprises a metal from Groups 3-12 of the Periodic Table, or combinations thereof;
wherein M²⁺ comprises divalent magnesium, calcium, strontium, barium, or combinations thereof; and
wherein M⁴⁺ comprises tetravalent silicon, titanium, zirconium, or combinations thereof;
with the proviso that when M²⁺ is magnesium, M⁴⁺ is not titanium or zirconium.

2. The catalyst of claim 1 wherein M comprises Cu, Co, Fe, Ni, Ru, Rh, Pd, Ir, Pt, Ag, Au, or combinations thereof.

3. The catalyst of claim 1 wherein M comprises Cu.

4. The catalyst of claim 1 wherein M is present in an amount of 0.01 wt % to 50 wt %.

5. The catalyst of claim 1 wherein M²⁺ comprises divalent magnesium.

6. The catalyst of claim 1 wherein M²⁺ O is present in an amount of 1 wt % to 98 wt %.

7. The catalyst of claim 1 wherein M⁴⁺ comprises silicon.

8. The catalyst of claim 1 wherein M⁴⁺O₂ is present in an amount of 1 wt % to 98 wt %.

9. The catalyst of claim 1 further comprising:
a salt or oxide of a metal from Group 1 of the Periodic Table, or combinations thereof.

10. The catalyst of claim 9 wherein the Group 1 metal comprises Li, Na, K, Rb, Cs, or combinations thereof.

11. The catalyst of claim 9 wherein the Group 1 metal is present in an amount of 0.01 wt % to 10 wt %.

12. A method of producing isobutanol comprising:
reacting ethanol or propanol with methanol in the presence of an alcohol condensation catalyst under reaction conditions to produce isobutanol;
wherein the alcohol condensation catalyst comprises a M/M²⁺M⁴⁺ oxide catalyst;
wherein M comprises metals from Groups 3-12 of the Periodic Table, or combinations thereof;
wherein M²⁺ comprises divalent magnesium, calcium, strontium, barium, or combinations thereof;
wherein M⁴⁺ comprises tetravalent silicon, titanium, zirconium, or combinations thereof.

13. The method of claim 12 wherein the reaction conditions comprise one or more of:
a temperature in a range of 100° C. to 500° C.; or
a pressure in a range of 5 kPa to 30,000 kPa.

14. The catalyst of claim 12 wherein M comprises Cu, Co, Fe, Ni, Ru, Rh, Pd, Ir, Pt, Ag, Au, or combinations thereof.

15. The catalyst of claim 12 wherein M is present in an amount of 0.01 wt % to 50 wt %.

16. The catalyst of claim 12 wherein M²⁺ O is present in an amount of 1 wt % to 98 wt %.

17. The catalyst of claim 12 wherein M⁴⁺O₂ is present in an amount of 1 wt % to 98 wt %.

18. The catalyst of claim 12 further comprising:
a salt or oxide of a metal from Group 1 of the Periodic Table, or combinations thereof.

19. The catalyst of claim 18 wherein the Group 1 metal is present in an amount of 0.01 wt % to 10 wt %.
